(19) United States
(12) Patent Application Publication
(10) Pub. No.: US 2019/0038632 A1
(43) Pub. Date: Feb. 7, 2019

(54) COPANLISIB BIOMARKERS

(71) Applicant: BAYER PHARMA AKTIENGESELLSCHAFT, Berlin (DE)

(72) Inventors: Li LIU, East Hanover, NJ (US); Carol PENA, Basking Ridge, NJ (US); Jie CHENG, Belle Mead, NJ (US); Karl KÖCHERT, Potsdam (DE)

(73) Assignee: BAYER PHARMA AKTIENGESELLSCHAFT, Berlin (DE)

(21) Appl. No.: 16/074,728

(22) PCT Filed: Jan. 30, 2017
(86) PCT No.: PCT/EP2017/051903

§ 371 Date: Aug. 1, 2018

Related U.S. Application Data
(60) Provisional application No. 62/289,713, filed on Feb. 1, 2016.
(60) Provisional application No. 62/376,017, filed on Aug. 17, 2016.

(51) Int. Cl. A61K 31/5377; A61P 35/00; C12Q 1/6886; G06F 19/20

(52) U.S. Cl. CPC A61K 31/5377; A61P 35/00; C12Q 2600/106; G06F 19/20; C12Q 1/6886

(57) ABSTRACT

This invention provides biomarkers based on the gene expression profiling which can discriminate between patients who response to and/or with longer progression free survival and patients who do not response to and/or with shorter progression free survival from copanlisib treatment in lymphoma including indolent and aggressive NHLs and CLLs. The present invention relates to the use of genes from the BCR, PI3K, NFkB, IL6, inflammation and stromal processes as predictive biomarkers for various human cancers including but not limited to NHLs.

### INTRODUCTION

The present invention relates to gene expression markers and signatures of copanlisib therapy and to methods of use in cancer: it provides biomarkers based on the gene expression profiling which can discriminate between patients who response to and/or with longer progression free survival, and patients who do not response to and/or with shorter progression free survival from copanlisib treatment in lymphoma including indolent and aggressive non-Hodgkin's lymphoma (hereinafter referred to as “NHL”) and chronic lymphocytic leukemiae (hereinafter referred to as “CLLs”). The present invention relates to the use of genes from the BCR, PI3K, NFkB, IL6, inflammation and stromal processes as predictive biomarkers for various human cancers including but not limited to NHLs.

### BACKGROUND

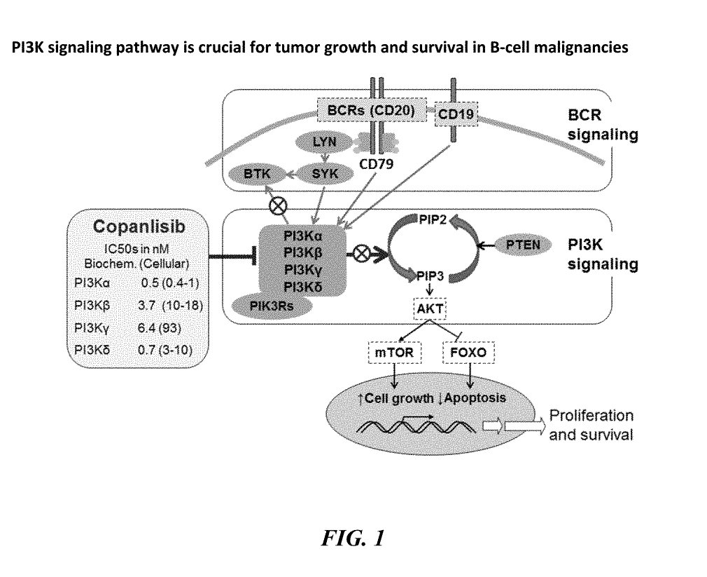

Copanlisib, a novel pan-class I PI3K inhibitor with predominant activity against a and 5 isoforms, showed promising single agent anti-tumor activity in a phase 2 study in heavily pretreated patients with indolent and aggressive NHL. This mechanism of action (vide FIG. 1) has been shown to translate into antitumor activity in NHL. Identification of biomarkers that predict sensitivity to copanlisib could result in more effective biomarker-driven targeted therapy for cancer. Although activating mutations in PIK3CA and/or alterations in PTEN have been shown to be determinants of sensitivity, whereas RAS/RAF mutations/activation result in resistance to PI3K pathway inhibition in preclinical models, no predictive markers for PI3K inhibitors have been defined yet in the clinical setting.

We use tumor gene expression profiling and bioinformatic analyses to identify novel genes or pathways associated with tumor response and benefit/outcomes from copanlisib treatment and provide predictive markers and rationales for novel combination considerations.

### DETAILED DESCRIPTION OF THE INVENTION

### Definitions of Terms Used in the Context of the Present Invention

The term “compound” as used herein means copanlisib, particularly a pharmacologically acceptable salt thereof, in particular copanlisib dihydrochloride, i.e. the active ingredient or active substance which is administered to the subject for the treatment of the “indication” as defined herein.

The term “indication” as used herein means the cancer type or tumor type for which it was found that subjects having this cancer type are likely to be responder to the therapy with the compound if the cancer is characterized by the stratification feature as defined herein. The indication is characterized by the “stratification feature” as defined herein. The indication as used herein is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”).

The term “stratification feature” as used herein is the feature of the subject's cancer type recommending the treatment with the active ingredient. The feature is an up-regulation of one or more genes (also referred to as “specific markers”) which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;

and/or
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

The term “up-regulation” (also referred to as “high expression”) or “down-regulation” (also referred to as “low expression”) of a gene or a gene signature or a gene pathway as defined herein is determined by comparing the expression level of a gene from measurement or a score (for example WGS) calculated/derived from the expression levels of all genes in the signature or pathway, with a threshold value (also referred to as a “cut off”). A cut off can be a median value generated from the tumors collected from the indication of the disease or established from a clinical trial evaluating the relationship between the expression level of a gene or a gene signature score and efficacy by the treatment of copanlisib. Up-regulation (or high expression): higher than a threshold (a cut off). Down-regulation (or low expression): lower than a threshold (a cut off).

The term “stratification feature is determined positively” means that the presence of said stratification feature (which can be also a decrease or absence of said specific marker) was confirmed.

The term “stratification method” means the method by which said stratification feature is determined, e.g. a stratification method wherein the expression levels of said genes and gene signatures (patterns) are determined by a method such as: Affymetrix array, RT-PCR, RNAseq, nanostrings, RNAscope, for example, the degree of said up-regulation being statistically verified using the median weighted gene expression scores (WGS) for genes within the specific pathways and median gene expression signal level of affymetrix array for single genes as cut off values.

The term “sample” as used herein means the sample which is used in the stratification method, e.g. tumor sample, tissue sample, biological sample, blood sample, particularly tumor tissue of tumor cells.

Within the context of the present invention, the stratification feature can be used to characterize the indication or the subject, as defined herein.

The First Aspect of the Present Invention Relates to Biomarkers for Copanlisib Wherein the Indication is Characterized by the Stratification Feature:

In a first feature of the first aspect, the present invention relates to the use of copanlisib, particularly copanlisib dihydrochloride, for the preparation of a medicament for treating an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject, wherein said indication is characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18 gene.

In an embodiment of the first feature of the first aspect, the present invention relates to said use wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, for example, the degree of said up-regulation being statistically verified using the median weighted gene expression scores (WGS) for genes within the specific pathways and median gene expression signal level of affymetrix array for single genes as cutoff values.

In a second embodiment of the first feature of the first aspect, the present invention relates to said use wherein the subject who shall be treated is one for whom a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

has been determined positively in tumor tissue or tumor cells from the subject.

In a third embodiment of the first feature of the first aspect, the present invention relates to said use wherein the subject or the cancer of said subject is characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In a variant of the third embodiment of the first feature of the first aspect, the present invention relates to said use wherein the subject the cancer of said subject is characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a variant of an embodiment of the first feature of the first aspect, the present invention relates to the use of copanlisib, particularly copanlisib dihydrochloride, in the manufacture of a medicament for treating an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), wherein the subject has been determined to be a responder/stratified/identified by a method according to a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a second feature of the first aspect, the present invention relates to the use of copanlisib, particularly copanlisib dihydrochloride in the manufacture of a medicament for a method of treatment of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject, said method comprising the steps:
a) assaying tumor tissue or tumor cells from the subject by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values, and
b) determining if a stratification feature which is an up-regulation of one or more genes which is (are) predictive of a response to copanlisib, particularly copanlisib dihydrochloride, and/or progression free survival, and which is (are) selected from:
   BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
   PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
   GPR18;
and
c) administering a therapeutically effective amount of copanlisib, particularly copanlisib dihydrochloride,
   if said stratification feature is determined positively.

In a third feature of the first aspect, the present invention relates to a method for identifying a subject having an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) disposed to respond favorably to copanlisib, particularly copanlisib dihydrochloride, wherein the method comprises the detection of a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

in tumor tissue or tumor cells from the subject.

In a first embodiment of the third feature of the first aspect, the present invention relates to said method wherein the stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is detected in tumor tissue or tumor cells.

In a second embodiment of the third feature of the first aspect, the present invention relates to said method wherein said a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a fourth feature of the first aspect, the present invention relates to a method for identifying a subject having an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), who is more likely to respond to a therapy comprising copanlisib, particularly copanlisib dihydrochloride, the method comprising:
a) determining a stratification feature which is an up-regulation of one or more genes which is (are) predictive of a response to copanlisib, particularly copanlisib dihydrochloride, and/or progression free survival, and which is (are) selected from:
   BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
   PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
   GPR18;
   in-vitro in tumor tissue or tumor cells from said subject;
b) identifying the subject being more likely to respond to a therapy comprising copanlisib, particularly copanlisib dihydrochloride, when the stratification feature which is an up-regulation of one or more genes which is (are) predictive of a response to copanlisib, particularly copanlisib dihydrochloride, and/or progression free survival, and which is (are) selected from:
   BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
   PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
   GPR18;
   is present.

In a fifth feature of the first aspect, the present invention relates to a method for identifying a subject having an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), who is less likely to respond to a therapy comprising copanlisib, particularly copanlisib dihydrochloride, the method comprising:
a) determining a stratification feature which is an up-regulation of one or more genes which is (are) predictive of a response to copanlisib, particularly copanlisib dihydrochloride, and/or progression free survival, and which is (are) selected from:
   BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
   PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
   GPR18;
in tumor tissue or tumor cells from said subject;
b) identifying the subject being less likely to respond to a therapy comprising copanlisib, particularly copanlisib dihydrochloride, when said stratification feature is absent.

In a sixth feature of the first aspect, the present invention relates to the use of tumor tissue or tumor cells for stratifying an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), from a subject disposed to respond favorably to copanlisib, particularly copanlisib dihydrochloride.

In a seventh feature of the first aspect, the present invention relates to a method of predicting/determining whether a subject having an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) will be non-responsive/responsive/will respond to the treatment with copanlisib, particularly copanlisib dihydrochloride, wherein the method comprises the detection of a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;
in tumor tissue or tumor cells from the subject.

In an embodiment of the seventh feature of the first aspect, the present invention relates to said method wherein the sample is tumor tissue or tumor cells.

In a second embodiment of the seventh feature of the first aspect, the present invention relates to said method wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In an eighth feature of the first aspect, the present invention relates to a method of determining the likelihood that an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) subject benefits from treatment with copanlisib, particularly copanlisib dihydrochloride, said the method comprising:
a) determining a stratification feature which is an up-regulation of one or more genes which is (are) predictive of a response to copanlisib, particularly copanlisib dihydrochloride, and/or progression free survival, and which is (are) selected from:
   BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
   PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
   GPR18;
   in-vitro in tumor tissue or tumor cells from said subject
b) identifying the subject being more likely to respond to a therapy comprising copanlisib, particularly copanlisib dihydrochloride, when said stratification feature is determined positively.

In a ninth feature of the first aspect, the present invention relates to a method of optimizing therapeutic efficacy of treatment of a subject having an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) as more likely to respond to a therapy comprising copanlisib, particularly copanlisib dihydrochloride, said method comprising:
a) determining the presence of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by a stratification feature which is an up-regulation of one or more genes which is (are) predictive of a response to copanlisib, particularly copanlisib dihydrochloride, and/or progression free survival, and which is (are) selected from:
   BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
   PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
   GPR18;
   in tumor tissue or tumor cells of the subject,
b) identifying the subject as more likely to respond to a therapy comprising copanlisib, particularly copanlisib dihydrochloride, when said stratification feature is determined positively;
c) administering a therapeutically effective amount of copanlisib, particularly copanlisib dihydrochloride, if said stratification feature is determined positively;
d) determining if said stratification feature in tumor tissue or tumor cells of the subject having been administered therapeutically effective amount of copanlisib, particularly copanlisib dihydrochloride, is increased/decreased,
e) adopting treatment by lower/higher dosing, other dosage regimen, etc. . . .

In a tenth feature of the first aspect, the present invention relates to a method of monitoring treatment response in a subject with an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) treated with copanlisib, particularly copanlisib dihydrochloride, wherein the method comprises:
a) the detection of a stratification feature which is an up-regulation of one or more genes which is (are) predictive of a response to copanlisib, particularly copanlisib dihydrochloride, and/or progression free survival, and which is (are) selected from:
   BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
   PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
   GPR18;
in tumor tissue or tumor cells from said subject,
b) comparing said stratification feature to those from responder and non-responder, and
c) identifying whether the subject has a responder or non-responder pattern to determine whether the treatment is to be continued.

In an embodiment of the tenth feature of the first aspect, the present invention relates to said method wherein a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In an eleventh feature of the first aspect, the present invention relates to copanlisib, particularly copanlisib dihydrochloride, for the use in a method of treating non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;
in a subject.

In an embodiment of the eleventh feature of the first aspect, the present invention relates to copanlisib, particularly copanlisib dihydrochloride, wherein the characterization of the an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), is performed in tumor tissue or tumor cells (or corresponding tissue sample as applicable).

In a second embodiment of the eleventh feature of the first aspect, the present invention relates to copanlisib, particularly copanlisib dihydrochloride, wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a further embodiment of the eleventh feature of the first aspect, the present invention relates to copanlisib, particularly copanlisib dihydrochloride, for the use in a method of treating a subject diagnosed with an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;
   said method comprising the steps
   a) assaying tumor tissue or tumor cells from the subject by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values; and
   b) determining said stratification feature; and
   c) administering a therapeutically effective amount of copanlisib, particularly copanlisib dihydrochloride, if said stratification feature is determined positively.

In a twelfth feature of the first aspect, the present invention relates to the use of copanlisib, particularly copanlisib dihydrochloride, for the treatment and/or prophylaxis of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the twelfth feature of the first aspect, the present invention relates to said use wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a second embodiment of the twelfth feature of the first aspect, the present invention relates to said use wherein the subject who shall be treated is one for whom said stratification feature has been determined in tumor tissue or tumor cells from the subject.

In a thirteenth feature of the first aspect, the present invention relates to the use of a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

as stratification marker in the treatment of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) with copanlisib, particularly copanlisib dihydrochloride.

In an embodiment of the thirteenth feature of the first aspect, the present invention relates to said use wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a second embodiment of the thirteenth feature of the first aspect, the present invention relates to said use wherein the subject who shall be treated is one for whom said stratification feature has been determined in tumor tissue or tumor cells from said subject.

In a fourteenth feature of the first aspect, the present invention relates to a method for the treatment and/or prophylaxis of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

using an effective amount of copanlisib, particularly copanlisib dihydrochloride.

In an embodiment of the fourteenth feature of the first aspect, the present invention relates to said method of treatment wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a second embodiment of the fourteenth feature of the first aspect, the present invention relates to said method of treatment wherein the subject who shall be treated is one for whom said stratification feature has been determined in tumor tissue or tumor cells from the subject.

In a variant of the second embodiment of the fourteenth feature of the first aspect, the present invention relates to said method of treatment wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a fifteenth feature of the first aspect, the present invention relates to a method of treatment of a subject diagnosed with an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), comprising the steps:
a) assaying tumor tissue or tumor cells from the subject by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values; and
b) determining if a stratification feature which is an up-regulation of one or more genes which is (are) predictive of a response to copanlisib, particularly copanlisib dihydrochloride, and/or progression free survival, and which is (are) selected from:
   BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
   PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
   GPR18; and
c) administering a therapeutically effective amount of copanlisib, particularly copanlisib dihydrochloride, if said stratification feature is determined positively.

In a sixteenth feature of the first aspect, the present invention relates to a method of treating a subject suffering from an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”),

comprising administering a therapeutically effective amount of copanlisib, particularly copanlisib dihydrochloride, to the subject selected for said therapy based on a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

thereby treating said subject.

In a seventeenth feature of the first aspect, the present invention relates to a pharmaceutical combination comprising copanlisib, particularly copanlisib dihydrochloride, in combination with at least one or more further active substances for use in a method of treating and/or prophylaxis of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;
in a subject.

In an embodiment of the seventeenth feature of the first aspect, the present invention relates to said pharmaceutical combination wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a second embodiment of the seventeenth feature of the first aspect, the present invention relates to said pharmaceutical combination wherein said stratification feature is determined in tumor tissue or tumor cells from the subject.

In an eighteenth feature of the first aspect, the present invention relates to the use of a pharmaceutical combination comprising copanlisib, particularly copanlisib dihydrochloride, in combination with at least one or more further active substances for the preparation of a medicament for treating and/or prophylaxis of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”),

wherein a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined in tumor tissue or tumor cells from the subject.

In an embodiment of the eighteenth feature of the first aspect, the present invention relates to said use of a pharmaceutical combination wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a nineteenth feature of the first aspect, the present invention relates to a pharmaceutical formulation comprising copanlisib, particularly copanlisib dihydrochloride, in combination with an inert, nontoxic, and/or pharmaceutically suitable adjuvant for use in a method of treating and/or prophylaxis of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the nineteenth feature of the first aspect, the present invention relates to said pharmaceutical formulation wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a second embodiment of the nineteenth feature of the first aspect, the present invention relates to said pharmaceutical formulation wherein said stratification feature is determined in tumor tissue or tumor cells containing tumor cells from the subject.

In a third embodiment of the nineteenth feature of the first aspect, the present invention relates to the use of said pharmaceutical formulation comprising copanlisib, particularly copanlisib dihydrochloride, in combination with an inert, nontoxic, and/or pharmaceutically suitable adjuvant for (the manufacture of a medicament for) treating and/or prophylaxis of an indication which is non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), wherein a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined in tumor tissue or tumor cells from the subject.

In a variant of the third embodiment of the nineteenth feature of the first aspect, the present invention relates to said use of said pharmaceutical formulation wherein said stratification feature is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

The Second Aspect of the Present Invention Relates to Biomarkers for Copanlisib Wherein Subject is Characterized by the Stratification Feature:

In a first feature of the second aspect, the present invention relates to the use of copanlisib, particularly copanlisib dihydrochloride, for the preparation of a medicament for treating non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject, wherein said subject is selected by having a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the first feature of the second aspect, the present invention relates to said use wherein said stratification feature, which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN,
GPR18,

is determined by a stratification method wherein the expression levels of said genes and gene signatures (patterns) are determined by Affymetrix array, RT-PCR, RNAseq, nanostrings, RNAscope, for example the degree of said up-regulation being statistically verified using the median weighted gene expression scores (WGS) for genes within the specific pathways and median gene expression signal level of affymetrix array for single genes as cutoff values.

In a second embodiment of the first feature of the second aspect, the present invention relates to said use wherein said stratification feature, which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN,
GPR18,

has been determined positively in a sample which is tumor tissue or tumor cells of said subject, said up-regulation of said genes being determined and compared to the extent of expression in the pre-defined cutoff values.

In a second feature of the second aspect, the present invention relates to the use of copanlisib, particularly copanlisib dihydrochloride, for the manufacture of a medicament for a method of treatment of non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject, wherein said subject has been determined to be a responder, and has been stratified and has been selected, by a method comprising the steps:

a) assaying a sample which is tumor tissue or tumor cells of said subject by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values; and

b) determining said stratification feature, which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

and

c) administering a therapeutically effective amount of copanlisib, particularly copanlisib dihydrochloride if said stratification feature is determined positively.

In a third feature of the second aspect, the present invention relates to copanlisib, particularly copanlisib dihydrochloride, for the use in a method of treating non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject, characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the third feature of the second aspect, the present invention relates to copanlisib, particularly copanlisib dihydrochloride, wherein said characterization is performed in a tumor tissue or tumor cells from said subject.

In a second embodiment of the third feature of the second aspect, the present invention relates to copanlisib, particularly copanlisib dihydrochloride, wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a fourth feature of the second aspect, the present invention relates to copanlisib, particularly copanlisib dihydrochloride, for the use in a method of treating non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject, characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

said method comprising the steps:

a) assaying a sample which is tumor tissue or tumor cells of said subject by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values; and

b) determining said stratification feature, which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

and

c) administering a therapeutically effective amount of copanlisib, particularly copanlisib dihydrochloride if said stratification feature is determined positively.

In a fifth feature of the second aspect, the present invention relates to the use of copanlisib, particularly copanlisib dihydrochloride, for the treatment and/or prophylaxis of non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject characterized by a stratification feature which is an up-regulation of one or more genes which is (are) predictive of a response to copanlisib, particularly copanlisib dihydrochloride, and/or progression progress free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the fifth feature of the second aspect, the present invention relates to said use wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B; PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a second embodiment of the fifth feature of the second aspect, the present invention relates to said use wherein said stratification feature, which is an up-regulation of one or more genes which is (are) predictive of a response to copanlisib, particularly copanlisib dihydrochloride, and progress free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

has been determined in tumor tissue or tumor cells from said subject.

In a sixth feature of the second aspect, the present invention relates to a method for the treatment and/or prophylaxis of non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

using an effective amount of copanlisib, particularly copanlisib dihydrochloride.

In an embodiment of the sixth feature of the second aspect, the present invention relates to said method of treatment wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a second embodiment of the sixth feature of the second aspect, the present invention relates to said method of treatment wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

has been determined in tumor tissue or tumor cells from said subject.

In a variant of the second embodiment of the sixth feature of the second aspect, the present invention relates to said method of treatment wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a seventh feature of the second aspect, the present invention relates to a pharmaceutical combination comprising copanlisib, particularly copanlisib dihydrochloride, in combination with at least one or more further active substances for use in a method of treating and/or prophylaxis of non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the seventh feature of the second aspect, the present invention relates to said pharmaceutical combination wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a second embodiment of the seventh feature of the second aspect, the present invention relates to said pharmaceutical combination wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a further embodiment of the seventh feature of the second aspect, the present invention relates to said pharmaceutical combination wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

has been determined in tumor tissue or tumor cells from said subject.

In an eighth feature of the second aspect, the present invention relates to the use of a pharmaceutical combination comprising copanlisib, particularly copanlisib dihydrochloride, in combination with at least one or more further active substances for the preparation of a medicament for treating and/or prophylaxis of non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the eighth feature of the second aspect, the present invention relates to said use of the pharmaceutical formulation wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a second embodiment of the eighth feature of the second aspect, the present invention relates to said use of the pharmaceutical formulation wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined in tumor tissue or tumor cells from said subject.

In a ninth feature of the second aspect, the present invention relates to a pharmaceutical formulation comprising copanlisib, particularly copanlisib dihydrochloride, in combination with an inert, nontoxic, and/or pharmaceutically suitable adjuvant for use in a method of treating and/or prophylaxis of non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the ninth feature of the second aspect, the present invention relates to said pharmaceutical formulation wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B; PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a second embodiment of the ninth feature of the second aspect, the present invention relates to said pharmaceutical formulation wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

has been determined in tumor tissue or tumor cells from said subject.

In a further embodiment of the ninth feature of the second aspect, the present invention relates to said pharmaceutical formulation wherein stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

has been determined in tumor tissue or tumor cells from said subject.

In a tenth feature of the second aspect, the present invention relates to the use of a pharmaceutical combination comprising copanlisib, particularly copanlisib dihydrochloride, in combination with at least one or more further active substances for the preparation of a medicament for treating and/or prophylaxis of non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”) in a subject characterized by a stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18.

In an embodiment of the tenth feature of the second aspect, the present invention relates to said use of a pharmaceutical formulation wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined by a stratification method wherein the expression levels of said genes and gene signatures (patterns) are determined by Affymetrix array, RT-PCR, RNAseq, nanostrings, RNAscope, the degree of said up-regulation being statistically verified using the median weighted gene expression scores (WGS) for genes within the specific pathways and median gene expression signal level of affymetrix array for single genes as cutoff values.

In a second embodiment of the tenth feature of the second aspect, the present invention relates to said use of a pharmaceutical formulation wherein said stratification feature which is an up-regulation of one or more genes which is (are) predictive of a response to copanlisib, particularly copanlisib dihydrochloride, and/or progression free survival, and which is (are) selected from:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN;
GPR18;

is determined in tumor tissue or tumor cells from said subject.

In a further feature of the first aspect or the second aspect, the present invention relates to a test for a gene expression signature measuring:
an up-regulation of one or more BCR pathway genes selected from: BLNK, BTK, CD19, CD22, CD40, CD69, CD72, CD79A, CD79B, LYN, MS4A1 (CD20), SYK, TNFRSF13B, TNFRSF17, FCGR2B,

to characterize BCR signaling in a subject to assess the suitability of said subject to a treatment with copanlisib, particularly copanlisib dihydrochloride,

said test being the measurement of said genes and gene signatures (patterns) using one of the following methods, for example: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), It being possible for said cut off to be a median value generated from the tumors collected from the indication of the disease or established from a clinical trial evaluating the relationship between the expression level of a gene or a gene signature score and efficacy by the treatment of copanlisib.

In a further feature of the first aspect or the second aspect, the present invention relates to a test for a gene expression signature measuring:
an up-regulation of one or more PI3K pathway genes selected from: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN,

to characterize PI3K signaling in a subject to assess the suitability of said subject to a treatment with copanlisib, particularly copanlisib dihydrochloride,

said test being the measurement of said genes and gene signatures (patterns) using one of the following methods, for example: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), It being possible for said cut off to be a median value generated from the tumors collected from the indication of the disease or established from a clinical trial evaluating the relationship between the expression level of a gene or a gene signature score and efficacy by the treatment of copanlisib.

In a further feature of the first aspect or the second aspect, the present invention relates to a test for a gene expression signature measuring:
an up-regulation of one or more BCR pathway genes selected from: BLNK, BTK, CD19, CD22, CD40, CD69, CD72, CD79A, CD79B, LYN, MS4A1 (CD20), SYK, TNFRSF13B, TNFRSF17, FCGR2B;

and
an up-regulation of one or more PI3K pathway genes selected from: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN,

to characterize BCR and PI3K signaling in a subject to assess the suitability of said subject to a treatment with copanlisib, particularly copanlisib dihydrochloride,

said test being the measurement of said genes and gene signatures (patterns) using one of the following methods, for example: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), It being possible for said cut off to be a median value generated from the tumors collected from the indication of the disease or established from a clinical trial evaluating the relationship between the expression level of a gene or a gene signature score and efficacy by the treatment of copanlisib.

In a further feature of the first aspect or the second aspect, the present invention relates to a kit for the selection of a subject suffering from non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by and determining a stratification feature according to any embodiment of the first aspect or the second aspect as defined above.

In an embodiment of said further feature of the first aspect or the second aspect, the present invention relates to said kit wherein said treatment is a monotherapy or combination therapy.

In a second embodiment of said further feature of the first aspect or the second aspect, the present invention relates to said kit wherein said stratification feature is determined in a sample of tumor tissue or tumor cells from said subject.

In an additional feature of the first aspect or the second aspect, the present invention relates to a test for a gene expression signature measuring:
an up-regulation of one or more genes selected from: AKT1, AKT3, BLNK, BTK, CD19, CD69, CD72, CD79B, CD8B, FCGR2B, GAB1, LYN, MS4A1, PIK3AP1, PIK3C3, PIK3CA, PIK3CB, PIK3CD, PIK3CG, PIK3R1, PIK3R3, PIK3R4, PIK3R5, PTEN, TLR4, TNFRSF13B, VIM, GPR18,

in a subject to assess the suitability of said subject to a treatment with copanlisib, particularly copanlisib dihydrochloride,

said test being the measurement of said genes and gene signatures (patterns) using one of the following methods, for example: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), It being possible for said cut off to be a median value generated from the tumors collected from the indication of the disease or established from a clinical trial evaluating the relationship between the expression level of a gene or a gene signature score and efficacy by the treatment of copanlisib.

In an additional feature of the first aspect or the second aspect, the present invention relates to a test for a gene expression signature measuring:
a down-regulation of one or more genes selected from: CD4, CD22, VCAN, TNFRSF17, NDE1, ICAM1, PIK3IP1, NFKBIA, MMP9, CD40, IL411, AKT2, CD79A, JAK3, MET, COL1A1, VWF, VEGFA, SPARC, FN1, SGK1, MT2A, IL1B, CD93, STAT5A, KDR, THBS1, LUM, S100A8, SERPINH1, BATF, PTGIR, CSTB, ITGB2, S100A9, SYK, CD14, CEBPB, SPHK1, NOP10, THBS2, COL5A2,

in a subject to assess the suitability of said subject to a treatment with copanlisib, particularly copanlisib dihydrochloride,

said test being the measurement of said genes and gene signatures (patterns) using one of the following methods, for example: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), It being possible for said cut off to be a median value generated from the tumors collected from the indication of the disease or established from a clinical trial evaluating the relationship between the expression level of a gene or a gene signature score and efficacy by the treatment of copanlisib.

In an additional feature of the first aspect or the second aspect, the present invention relates to a test for a gene expression signature measuring:
an up-regulation of one or more genes selected from: AKT1, AKT3, BLNK, BTK, CD19, CD69, CD72, CD79B, CD8B, FCGR2B, GAB1, LYN, MS4A1, PIK3AP1, PIK3C3, PIK3CA, PIK3CB, PIK3CD, PIK3CG, PIK3R1, PIK3R3, PIK3R4, PIK3R5, PTEN, TLR4, TNFRSF13B, VIM, GPR18,

and
a down-regulation of one or more genes selected from: CD4, CD22, VCAN, TNFRSF17, NDE1, ICAM1, PIK3IP1, NFKBIA, MMP9, CD40, IL411, AKT2, CD79A, JAK3, MET, COL1A1, VWF, VEGFA, SPARC, FN1, SGK1, MT2A, IL1B, CD93, STAT5A, KDR, THBS1, LUM, S100A8, SERPINH1, BATF, PTGIR, CSTB, ITGB2, S100A9, SYK, CD14, CEBPB, SPHK1, NOP10, THBS2, COL5A2,

in a subject to assess the suitability of said subject to a treatment with copanlisib, particularly copanlisib dihydrochloride,

said test being the measurement of said genes and gene signatures (patterns) using one of the following methods, for example: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), It being possible for said cut off to be a median value generated from the tumors collected from the indication of the disease or established from a clinical trial evaluating the relationship between the expression level of a gene or a gene signature score and efficacy by the treatment of copanlisib.

In an additional feature of the first aspect or the second aspect, the present invention relates to a kit for the selection of a subject suffering from non-Hodgkin's lymphoma (hereinafter abbreviated to “NHL”), particularly relapsed/refractory, indolent or aggressive non-Hodgkin's lymphoma (NHL), in particular follicular lymphoma (hereinafter abbreviated to “FL”), chronic lymphocytic leukaemia (hereinafter abbreviated to “CLL”), marginal zone lymphoma (hereinafter abbreviated to “MZL”), diffuse large B-cell lymphoma (hereinafter abbreviated to “DLBCL”), mantle cell lymphoma (MCL), transformed lymphoma (hereinafter abbreviated to “TL”), or peripheral T-cell lymphoma (hereinafter abbreviated to “PTCL”), characterized by and determining a stratification feature defined in any emdodiment of an additional feature of the first aspect or the second aspect defined above.

In an embodiment of the additional feature of the first aspect or the second aspect the present invention relates to said kit wherein said treatment is a monotherapy or combination therapy.

In a second embodiment of the additional feature of the first aspect or the second aspect the present invention relates to said wherein said stratification feature is determined in a sample of tumor tissue or tumor cells from said subject.

Pharmaceutical Formulations of Copanlisib of the Present Invention

As mentioned above, the present invention relates to copanlisib, particularly copanlisib dihydrochloride, which may be in the form of a pharmaceutical formulation which is ready for use to be administered simultaneously, concurrently, separately or sequentially. The components may be administered independently of one another by the oral, intravenous, topical, local installations, intraperitoneal or nasal route.

Said formulations or compositions can be utilized to achieve the desired pharmacological effect by administration to a patient in need thereof. A patient, for the purpose of this invention, is a mammal, including a human, in need of treatment for the particular condition or disease. Therefore, the present invention includes copanlisib, particularly copanlisib dihydrochloride, which is in the form of a pharmaceutical formulation composition that is comprised of a pharmaceutically acceptable carrier and a pharmaceutically effective amount of a said copanlisib, particularly copanlisib dihydrochloride. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of component, and/or combination. A pharmaceutically effective amount of a combination is preferably that amount which produces a result or exerts an influence on the particular condition being treated. Copanlisib, particularly copanlisib dihydrochloride, of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, copanlisib, particularly copanlisib dihydrochloride, can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatin type containing, for example, surfactants, lubricants, and inert fillers such as lactose, sucrose, calcium phosphate, and corn starch.

In another embodiment, copanlisib, particularly copanlisib dihydrochloride, of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

Copanlisib, particularly copanlisib dihydrochloride, of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions.

Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);

aerosol propellants (examples include but are not limited to carbon dioxide, CCl₂F₂, F₂ClC—CClF₂ and CCIF₃) air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile IV Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular suspension: The following solution or suspension can be prepared, for intramuscular injection:

50 mg/mL of the desired, water-insoluble compound of this invention

5 mg/mL sodium carboxymethylcellulose

4 mg/mL TWEEN 80

9 mg/mL sodium chloride

9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Method of Treating Cancer

Within the context of the present invention, the term “cancer” includes, but is not limited to, cancers of the breast, lung, brain, reproductive organs, digestive tract, urinary tract, liver, eye, skin, head and neck, thyroid, parathyroid and their distant metastases. Those disorders also include multiple myeloma, lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

The present invention relates to a method for using copanlisib, particularly copanlisib dihydrochloride, of the present invention, to treat cancer, as described infra, particularly mammalian NSCLC, CRC, melanoma, pancreatic cancer, hepatocyte or breast cancer. The salt of the present invention can be utilized to inhibit, block, reduce, decrease, etc., cell proliferation and/or cell division, and/or produce apoptosis, in the treatment or prophylaxis of cancer, in particular NSCLC, CRC, melanoma, pancreatic cancer, hepatocyte carcinoma or breast cancer. This method comprises administering to a mammal in need thereof, including a human, an amount of a combination of this invention, or a pharmaceutically acceptable salt, isomer, polymorph, metabolite, hydrate, solvate or ester thereof; etc. which is effective for the treatment or prophylaxis of cancer, in particular NSCLC, CRC, melanoma, pancreatic cancer, hepatocyte carcinoma or breast cancer.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Dose and Administration

Based upon standard laboratory techniques known to evaluate compounds useful for the treatment or prophylaxis of cancer, in particular NSCLC, CRC, melanoma, pancreatic cancer, hepatocyte carcinoma or breast cancer, by standard toxicity tests and by standard pharmacological assays for the determination of treatment of the conditions identified above in mammals, and by comparison of these results with the results of known medicaments that are used to treat these conditions, the effective dosage of the salt of this invention can readily be determined for treatment of the indication. The amount of the active ingredient to be administered in the treatment of the condition can vary widely according to many considerations, including, but not limited to the particular combination and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1,500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

The specific initial and continuing dosage regimen for each patient will vary according to the nature and severity of the condition as determined by the attending diagnostician, the activity of the specific combination employed, the age and general condition of the patient, time of administration, route of administration, rate of excretion of the drug, drug salts, and the like. The desired mode of treatment and number of doses of a combination of the present invention or a pharmaceutically acceptable salt or ester or composition thereof can be ascertained by those skilled in the art using conventional treatment tests.

Therapies Using the Salt of the Present Invention: One or More Further Pharmaceutical Agents.

Copanlisib, particularly copanlisib dihydrochloride, of the present invention can be administered as the sole pharmaceutical agent or in combination with one or more further active ingredient where the resulting combination of the salt of the present invention and the further active ingredient causes no unacceptable adverse effects. For example, copanlisib, particularly copanlisib dihydrochloride, of the present invention can be combined with a further active ingredient such as known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and salts thereof.

Said further active ingredient may be selected from the following:

131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Generally, the use of cytotoxic and/or cytostatic agents as further active ingredient in combination with copanlisib, particularly copanlisib dihydrochloride, of the present invention will serve to:

(1) yield better efficacy in reducing the growth of a tumor or even eliminate the tumor as compared to administration of either agent alone,

(2) provide for the administration of lesser amounts of the administered chemotherapeutic agents,

(3) provide for a chemotherapeutic treatment that is well tolerated in the patient with fewer deleterious pharmacological complications than observed with single agent chemotherapies and certain other combined therapies,

(4) provide for treating a broader spectrum of different cancer types in mammals, especially humans,

(5) provide for a higher response rate among treated patients,

(6) provide for a longer survival time among treated patients compared to standard chemotherapy treatments,

(7) provide a longer time for tumor progression, and/or

(8) yield efficacy and tolerability results at least as good as those of the agents used alone, compared to known instances where other cancer agent salts produce antagonistic effects.

### EXPERIMENTAL SECTION

Copanlisib and copanlisib dihydrochloride can be synthesised as described in European patent application number EP 11 161 111.7, and in PCT application number PCT/EP2012/055600 published under WO 2012/136553, both of which are hereby incorporated herein in their entirety by reference.

### Methods:

Individual formalin-fixed, paraffin-embedded (FFPE) samples from baseline tumor lesions of NHL patients that underwent copanlisib therapy in clinical study A were subjected to RNA gene expression studies on Affymetrix Gene ST 1.0 arrays by AltheaDx Inc. (San Diego, Calif., USA). Best response status and progression-free survival (hereinafter referred to as “PFS”) from independent review assessment were used for gene expression analysis. A total number of 24 patients including 3 complete responders, 1 confirmed (hereinafter referred to as “CR”) and 2 unconfirmed (hereinafter referred to as “CRu”) and 5 partial responders (hereinafter referred to as “PR”), 11 stable diseases (hereinafter referred to as “SD”) and 5 progressive diseases (hereinafter referred to as “PD”) with follicular lymphoma (hereinafter referred to as “FL”), marginal zone lymphoma (hereinafter referred to as “MZL”), mantle cell lymphoma (“MCL”), diffuse large B-cell lymphoma (hereinafter referred to as “DLBCL”), transformed indolent lymphoma or CLL who had baseline RNA expression profiling data of sufficient quantity and quality were used for gene expression analysis as listed in Table 1 (given in the experimental section).

Bioinformatics and statistical analyses were done for all 24 NHL patients. Both gene set enrichment analysis (hereinafter referred to as “GSEA”) (ref 1) and a single gene multivariate adaptive two way filtering approach (ref 2) were used to identify potential predictive markers and common signaling pathways associated with copanlisib response in lymphoma.

GSEA (http://www.broadinstitute.org/gsea/index.jsp), a computational method that determines whether an a priori defined set of genes shows statistically significant, concordant differences between two biological states (e.g. phenotypes, in this case based on clinical outcomes), was used to identify common signaling pathways associated with copanlisib response or lack of response in lymphoma. For GSEA, 34 gene sets (vide Tables 2 and 3) sharing key biological function/process in apoptosis, B-cell receptor (hereinafter referred to as “BCR”) signaling, IL6/JAK/STAT3, cytokine/chemokine, MAPK, MYC, MYD88, NFAT, NFkB, NOTCH, PI3K or tumor microenvironment (refs 3 and 4) were selected and generated. The magnitude of normalized enrichment scores (hereinafter referred to as “NES”) and false discovery rate (hereinafter referred to as “FDR”) q values were computed to evaluate the effectiveness of each gene set in identifying top candidates that influence copanlisib response/lack of response in the study. Gene sets were ranked for association with response based on NES values (where the higher the positive numbers, the more likely the patient would show response to copanlisib) associated with tumor response on copanlisib treatment, and low FDR q values (indicating lower likelihood of an association by random chance). In contrast, gene sets were ranked for association with lack of response based on negative NES values (where the lower the negative numbers, the more likely the patient would show lack of response to copanlisib), and low FDR q values (indicating lower likelihood of an association by random chance).

TABLE 2
Novel pathway genes.

| Apo./prolif. | BAD | BAX | BBC3 | BCL2 | BCL2L11 | BID | BIK | CCND1 | CCND3 |
|---|---|---|---|---|---|---|---|---|---|
| BCR | BLNK | BTK | CD19 | CD22 | CD40 | CD69 | CD72 | CD79A | CD79B |
| Cyto/ | IL8 | IL6 | IL1B | IL18 | CXCR5 | CXCR4 | CXCL9 | CXCL6 | CXCL2 |
| Chemo. | | | | | | | | | |
| Immune T- | CD80 | TNFRSF9 | TNFRSF4 | PDCD1 | IFNG | CTLA4 | CD274 | IL2RA | CD8B |
| cells | | | | | | | | | |
| MYC | TMEM97 | FGD6 | ZBED2 | EMP1 | TAF4B | NOP16 | LTBP2 | PES1 | SLC19A1 |
| | NOLC1 | MYC | COL8A2 | NGFR | TCOF1 | FARSA | SMTN | AIMP2 | NLRP1 |
| | ATP2B4 | VAMP1 | YPEL3 | IRF9 | FAM100A | CCDC86 | HIST1H2AC | DGKA | AHNAK |
| NFKB | SGK1 | BATF3 | CD14 | ICAM1 | BCL2A1 | RASGRP1 | RASSF4 | MREG | TNF |
| | NFKB2 | TRAF1 | CLU | TNFAIP2 | BATF | IRF1 | LYPLA2 | LAT2 | SSTR2 |
| | CARD11 | BIRC3 | PTGER4 | TMSB4X | BTG1 | TMSB4X | PAX5 | TMSB4X | |
| NFAT | CAMK4 | PRKCH | FKBP8 | MAP3K8 | PRKCE | PRKCZ | NFATC1 | NFATC2 | SFN |
| PI3K | AKT1 | AKT2 | AKT3 | GAB1 | PIK3AP1 | PIK3C3 | PIK3CA | PIK3CB | PIK3CD |
| Stromal | COL1A1 | COL5A2 | FN1 | MMP9 | S100A8 | S100A9 | SPARC | THBS1 | THBS2 |
| *Stromal | ADAM12 | BGN | CEBPA | COL13A1 | COL16A1 | COL1A1 | COL1A2 | COL5A1 | COL5A2 |
| signature 1 | EFEMP2 | EMP2 | FAP | FBN1 | FN1 | GPNMB | HSPG2 | IL1R1 | ITGB2 |
| | MFAP2 | MMP14 | MMP2 | MMP9 | PDGFC | PLAU | POSTN | SDC2 | SERPINH1 |
| *Stromal | ADH1B | ADIPOQ | CAV1 | CAV2 | CD93 | CXCL12 | ECSCR | EGFL7 | EHD2 |
| signature 2 | LAMB1 | LEPR | MMRN2 | PCDH18 | PECAM1 | PLIN1 | PTPRB | RBP4 | ROBO4 |
| #Immune | ACTN1 | ASAP2 | ATP8B2 | BIN2 | C1RL | CCSER2 | CD7 | CD8 | FAM46A |
| responsive | INPP1 | ITK | LEF1 | LGALS2 | LGALS3 | NFIC | NOL4L | PMEPA1 | PTRF |
| signature 1 | TNFRSF1B | TNFRSF25 | TNFSF12 | TNFSF13B | TTC39B | | | | |
| #Immune | BLVRA | SMG6 | C1QA | C1QB | C3AR1 | C4A | PXDC1 | HERC5 | DHRS3 |
| responsive | MITF | MRVI1 | NDN | OASL | PELO | SCARB2 | SEPT10 | TLR5 | |
| signature 2 | | | | | | | | | |

| Apo./prolif. | BAD | CDKN1B | MCL1 | MKI67 | PIK3IP1 | TNFSF10 | TP53 | TP53INP1 | [symbol] |
|---|---|---|---|---|---|---|---|---|---|
| BCR | BLNK | LYN | MS4A1 | SYK | TNFRSF13B | TNFRSF17 | FCGR2B | | |
| Cyto/ | IL8 | CXCL13 | CXCL12 | CXCL10 | CCR7 | CCL2 | CCL19 | IL4R | [symbol] |
| Chemo. | | | | | | | | | |
| Immune T- | CD80 | CD8A | CD7 | CD5 | CD4 | CD3G | CD3E | CD3D | [symbol] |
| cells | | | | | | | | | |
| MYC | TMEM97 | PLD6 | SERPINB1 | CDK4 | TTLL12 | MYBBP1A | TGM2 | PDLIM7 | [symbol] |
| | NOLC1 | PLEC1 | PHB | BOP1 | IMP4 | EXOSC5 | ZNF667 | CCDC137 | [symbol] |
| | ATP2B4 | SRM | DUSP2 | | | | | | |
| NFKB | SGK1 | STAT5A | C10orf10 | SIRPA | RAB7L1 | TNFAIP3 | IL4I1 | NFKB1 | [symbol] |
| | NFKB2 | GADD45B | MYD88 | C20orf27 | SGPP2 | KCNN4 | NDE1 | SOCS3 | N [symbol] |
| | CARD11 | | | | | | | | |
| NFAT | CAMK4 | PRKCB | BCL2L1 | | | | | | |
| PI3K | AKT1 | PIK3CG | PIK3IP1 | PIK3R1 | PIK3R2 | PIK3R3 | PIK3R4 | PIK3R5 | [symbol] |
| Stromal | COL1A1 | TLR4 | VEGFA | VIM | | | | | |
| *Stromal | ADAM12 | COL6A2 | COL6A3 | COL8A2 | CSF2RA | VCAN | CTGF | CYR61 | [symbol] |
| signature 1 | EFEMP2 | ITGAV | KITLG | LAMA4 | LAMB2 | LAMB3 | LOXL1 | LTBP2 | [symbol] |
| | MFAP2 | SPARC | TGFB1I1 | THBS1 | TIMP2 | | | | |
| *Stromal | ADH1B | ADGRL4 | ERG | FABP4 | ADGRF5 | GRB10 | IGFBP5 | ITGA9 | [symbol] |
| signature 2 | LAMB1 | SORBS1 | SPARCL1 | SPRY1 | TEK | TNXB | VWF | | |
| #Immune | ACTN1 | FLNA | FLT3LG | FNIP2 | GALNT12 | GNAQ | HCST | HOXB2 | [symbol] |
| responsive | INPP1 | RAB27A | RALGDS | SEMA4C | SEPW1 | SLC35A1 | STAT4 | TBC1D4 | [symbol] |
| signature 1 | TNFRSF1B | | | | | | | | |
| #Immune | BLVRA | DUSP3 | F8 | FCGR1A | GPBRC5B | HOXD8 | LGMN | ME1 | P [symbol] |
| responsive | MITF | | | | | | | | |
| signature 2 | | | | | | | | | |

#Wright et al, NEJM 2008-DLBCL,
*Dave et al, NEJM 2004-FL; [symbol]
indicates data missing or illegible when filed

TABLE 3
Pathways genes from GSEA MSigDB

| Pathways | ID |
|---|---|
| APOPTOSIS | KEGG_APOPTOSIS |
| BCR | BIOCARTA-BCR_PATHWAYS |
| BCR | KEGG_B_CELL_RECEPTOR_SIGNALING_PATHWAY |
| DLBCL | SHIPP_DLBCL_VS_FOLLICULAR_LYMPHOMA |
| IL6 | BIOCARTA_IL6_PATHWAY |
| IL6 | HALLMARK_IL6_JAK_STAT3_SIGNALING |
| INFLA | HALLMARK_INFLAMMATORY_RESPONSE |
| MAPK | KEGG_MAPK_SIGNALING_PATHWAY |
| MYC | COLLER_MYC_TARGETS |
| MYD88 | GNF2_MYD88 |
| MYD88 | REACTOME_MYD88_MAL_CASCADE_INITIATED_ON_PLASMA_MEMBRANE |
| NFAT | BIOCARTA_NFAT_PATHWAY |
| NFKB | BIOCARTA_NFKB_PATHWAYS |
| NFKB | HINATA_NFKB_IMMU_INF |
| NFKB | JAIN_NFKB_SIGNALING |
| NFKB | TIAN_TNF_SIGNALING_VIA_NFKB |
| NOTCH | KEGG_NOTCH_SIGNALING_PATHWAY |
| PTEN | BIOCARTA_PTEN_PATHWAY |
| PI3K | REACTOME_PI3K_AKT_ACTIVATION |
| TCR | KEGG_T_CELL_RECEPTOR_SIGNALING_PATHWAY |

A weighted gene expression score (hereinafter referred to as “WGS”) reflecting the overall expression level for each gene set was generated from logistic regression and Cox proportional hazards models to assess the association with response status (best-response-WGS) and PFS (PFS-WGS), respectively.

For pathways analysis, the WGS for a gene set of interest for a specific patient j was defined as follows:

$${WGS}_{j} = \sqrt{{\sum }_{i = 1}^{n} {( \frac{{\hat{β}}_{i}}{{SE}_{i}} * {mRNA}_{ij} )}^{2}}$$

i:=gene index [1, n:=gene set size]

j:=patient index

\hat{β}:=estimator for gene i dervied from the logistic regression (for response) and Cox (for PFS)models described above

SEᵢ, :=standard error for the estimation of \hat{β}ᵢ

mRNAᵢⱼ:=normalized gene expression of gene i inpatient j

For each gene set of interest, the association of the WGS with best response was estimated using the following WGS-model (pseudo-code representation), for PFS the model was adjusted to be a Cox proportional hazards model with the same predictors:

Responderᵢ˜logitLink(β₀+β₁*WGSᵢ+β₂*Ageᵢ+β₃*Sexᵢ+β₄*indicationGroupᵢ

i∈[1,number subjects]

Both the best-response-WGS as well as the PFS-WGS were in turn used in logistic regression or Cox regression models to assess the association of either WGS with the endpoints. Moreover, the raw best-response WGS was used to compute non-cross validated AUC estimates for responder (CR+PR) and non-responder (SD+PD) classifications.

In addition, adaptive two way filtering approach, that parsimoniously selects a small number of most informative genes was performed to identify any single genes associate with copanlisib response in the study (ref 2).

### Results:

The 24 analyzable patients including 3 CR, 5 PR, 11 SD, and 5 PD, with diagnoses of FL (n=10), MZL (n=2), MCL (n=2), DLBCL (n=5), transformed indolent (n=2) or CLL (n=3) are listed in Table 1. All the analysis was performed on the whole population of NHLs accounting for the indolent or aggressive nature of the disease type in multivariate models.

TABLE 1
Response status and PFS of the patients from clinical study A:

| | | | PFS | |
|---|---|---|---|---|
| | | Best Response | (Independent*) | Censor |
| Lymphoma Type | SUBJIDN | (Independent*) | Days | Status |
| CHRONIC | 160050003 | PR | 221 | 0 |
| LYMPHOCYTIC | 220020002 | PR | 681 | 0 |
| LEUKEMIA (N = 3) | 260020001 | SD | 222 | 0 |
| FOLLICULAR | 280040003 | CR | 499 | 1 |
| LYMPHOMA (N = 10) | 140030001 | CRu | 110 | 1 |
| | 220030001 | PR | 53 | 1 |
| | 240030001 | PR | 336 | 0 |
| | 120020003 | SD | 106 | 0 |
| | 120070001 | SD | 223 | 0 |
| | 120070002 | SD | 74 | 0 |
| | 140210001 | SD | 275 | 1 |
| | 240040001 | SD | 28 | 0 |
| | 280040001 | SD | 288 | 0 |
| MARGINAL ZONE | 260020002 | PR | 444 | 0 |
| LYMPHOMA (N = 2) | 100030003 | SD | 391 | 0 |
| DIFFUSE LARGE B- | 120030001 | SD | 56 | 1 |
| CELL LYMPHOMA | 160010004 | SD | 104 | 0 |
| (N = 5) | 240050001 | SD | 175 | 0 |
| | 100020001 | PD | 13 | 0 |
| | 280030001 | PD | 49 | 0 |
| MANTLE CELL | 160040001 | CRu | 377 | 0 |
| LYMPHOMA (N = 2) | 120020001 | PD | 7 | 0 |
| TRANS. INDOLENT | 120010001 | PD | 16 | 0 |
| LYMPHOMA (N = 2) | 160040004 | PD | 42 | 0 |

*data cutoff February 2015: Independent Assessment, for CLL-investigator assessment was used.
Responders: CR + CRu + PR = 8;
Non responders: SD + PD = 16
0 = not censored;
1 = censored

Gene signatures and pathways associated with copanlisib response status and PFS are listed in Table 4.

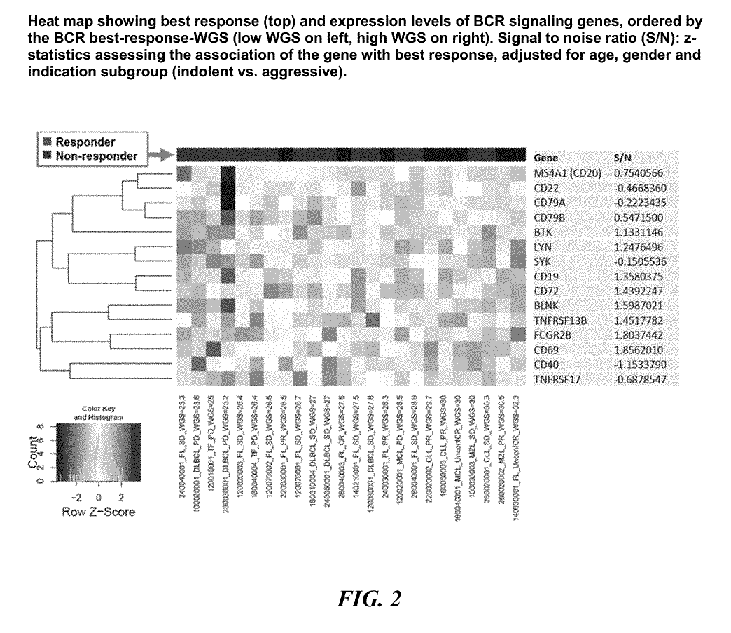

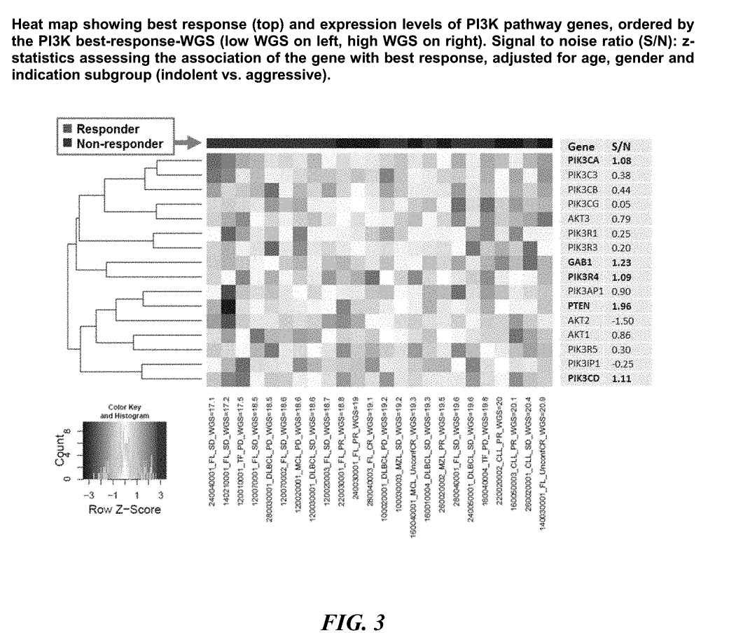

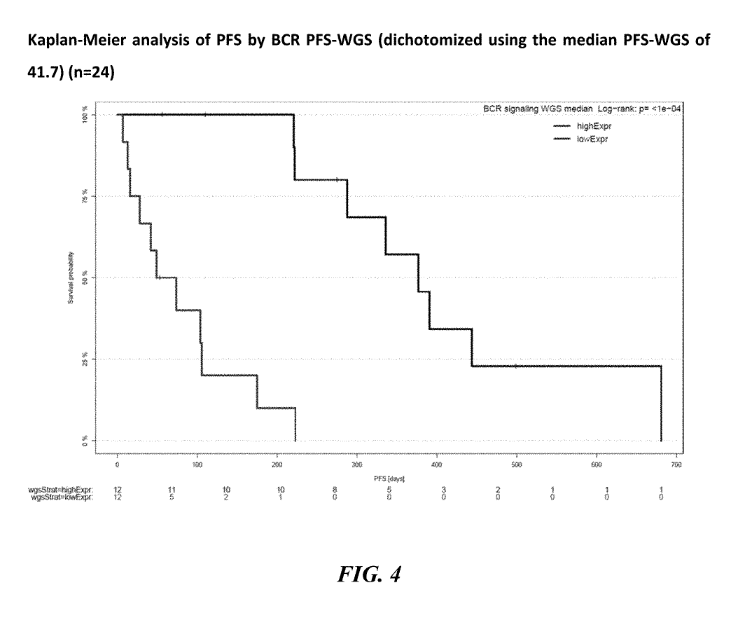

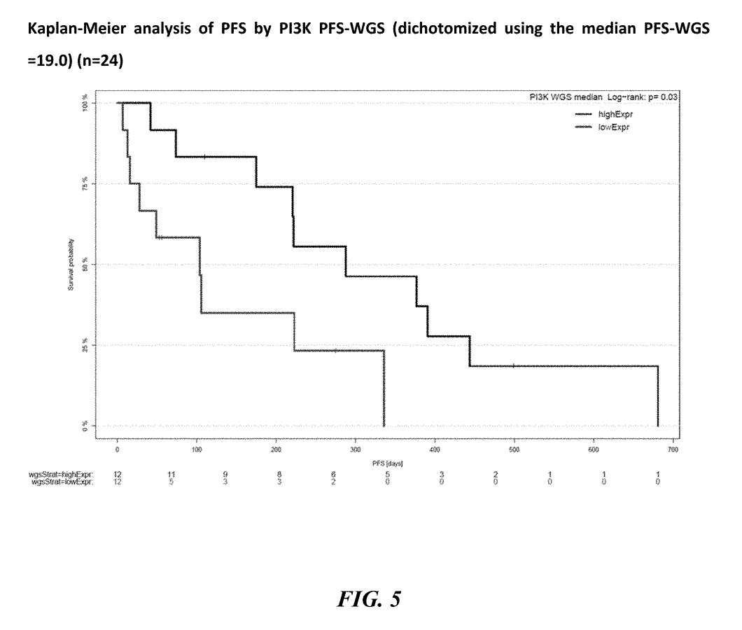

As shown in Table 4, among the full set of 24 NHLs, the top ranked gene sets associated with both objective responses (positive NES value≥1.4, FDRq≤0.17) and longer PFS (lack of progress, negative NES≤−1.4, FDRq≤0.12) identified by GSEA are those reflecting upregulated PI3K pathway and BCR signaling. For GESA response analysis, the representative BCR gene set (including CD19, CD20, BTK, and other genes shown in FIG. 2) and PI3K gene set (including PIK3CA, PIK3CB, PIK3CG, and PIK3CD, encoding the PI3K α, β, γ and δ catalytic subunits, respectively, and other genes shown in FIG. 3) have normalized enrichment scores (NES) of 1.92 and 1.62 with false discovery rates (FDR) of 0.014 and 0.087, respectively (Table 5). Accordingly, objective response rate is increased among patients with a high BCR pathway weighted gene expression score (WGS, reflecting overall expression level of the gene set) compared to low (nominal p=0.060, WGS based AUC=0.81) (Table 5 and FIG. 2), and in patients with high PI3K pathway WGS compared to low (nominal p=0.069, WGS based AUC=0.75) (Table 5 and FIG. 3). Using the median value of the WGS as a cut-off (BCR, WGSmedian=41.7; PI3K, WGS median=19.0), PFS is longer in copanlisib-treated NHL patients with high BCR WGS (377 vs 62 days, HR=0.035, nominal Cox model p<0.0001; FIG. 4) and in patients with high PI3K WGS (288 vs 104 days, HR=0.242, nominal Cox model p=0.022; FIG. 5) compared to those with low values.

TABLE 5
Gene expression profiling in clinical study A: Pathway gene sets
whose upregulation is associated with response (n = 24). For
the lists of genes included in each pathway gene set, see FIG. 2
and FIG. 3.

| | | | Best- | Best- |
|---|---|---|---|---|
| | GSEA | GSEA FDR | response-WGS | response-WGS |
| Pathway | NES | q value | p-value* | AUC* |
| BCR | 1.92 | 0.014 | 0.060 | 0.81 |
| PI3K | 1.62 | 0.087 | 0.069 | 0.75 |

GSEA, Gene set enrichment analysis;
NES, normalized enrichment score;
FDR, false discovery rate;
WGS, weighted gene expression score;
AUC, area under the curve;
BCR, B-cell receptor
*Based on assessing WGS association with best response while adjusting for gender, age and indication subgroup (indolent vs. aggressive)
**Using the WGS for ROC curve computation; no cross-validation was performed

On the other hand, among the full set of 24 NHLs, GSEA also identified the top ranked gene sets associated with lack of copanlisib response and/or with shorter PFS (Table 4). Gene sets/pathways involved in stromal/metastasis and inflammatory processes are associated with lack of copanlisib response (negative NES-1.77, FDRq≤0.01) and shorter PFS (progression positive NES value≥1.46, FDRq≤0.12). Both IL6/JAK/STAT3 and NFkB pathways are associated with lack of copanlisib response ((negative NES-1.45, FDRq≤0.1), however not with PFS.

In addition, using adaptive two way filtering approach, that parsimoniously selects a small number of most informative genes (ref 2), identified that high expression level of the GPR18 (G-protein coupled receptor 18) was potentially predictive of high likelihood of response with the training ROC AUC values of 0.95 and longer PFS with the concordance values of 0.73. When the gene expression values are dichotomized using the median expression level as a cut-off, a longer PFS was also observed in copanlisib-treated NHL patients with high (above the median) expression of GPR18 (377 vs. 106 days, HR=0.17, nominal Cox model p<0.01) when compared with patients who have low values. In contrast, high expression levels of MT2A (Metallothionein-2), NOP10 (NOP10 ribonucleoprotein and CSTB cystatin B (stefin B) genes were potentially predictive of lack of response with the training AUC 0.86 (0.71-1.0), 0.74 (0.54-0.94) and 0.85 (0.70-1.0) respectively and possibly with shorter PFS (Using a median gene expression level as cutoff, CSTB: HR=3.3 (0.9-12), p=0.07, median PFS days 74 and 288; ROC-AUC 0.85 (0.70-1.0); Concordance 0.713; NOP10: HR=1.9 (0.7-5.4) p=0.23 median PFS days 104 and 336 ROC-AUC 0.84 (0.67-1.0) Concordance 0.66; MT2A: HR=1.3 (0.8-2.2) p=0.30 median PFS days 106 and 336 ROC-AUC 0.76 (0.56-0.95); Concordance 0.66).

### REFERENCES

1. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: a knowledge-based approach for interpreting genome-wide expression profiles. Proc Natl Acad Sci USA. 2005 Oct. 25; 102(43):15545-50.
2. Cheng J. et al., An adaptive feature selection method for microarray data analysis, International conference of bioinformatics and biomedicine, 2012
3. Lenz G, Wright G, Dave S S, Xiao W, Powell J, Zhao H, Xu W, Tan B, Goldschmidt N, Iqbal J, Vose J, Bast M, Fu K, Weisenburger D D, Greiner T C, Armitage J O, Kyle A, May L, Gascoyne R D, Connors J M, Troen G, Holte H, Kvaloy S, Dierickx D, Verhoef G, Delabie J, Smeland E B, Jares P, Martinez A, Lopez-Guillermo A, Montserrat E, Campo E, Braziel R M, Miller T P, Rimsza L M, Cook J R, Pohlman B, Sweetenham J, Tubbs R R, Fisher R I, Hartmann E, Rosenwald A, Ott G, Muller-Hermelink H K, Wrench D, Lister T A, Jaffe E S, Wilson W H, Chan W C, Staudt L M, Stromal gene signatures in large-B-cell lymphomas. N Engl J Med. 2008 Nov. 27; 359(22):2313-23. doi: 10.1056/NEJMoa0802885.
4. Dave S S, Wright G, Tan B, Rosenwald A, Gascoyne R D, Chan W C, Fisher R I, Braziel R M, Rimsza L M, Grogan T M, Miller T P, LeBlanc M, Greiner T C, Weisenburger D D, Lynch J C, Vose J, Armitage J O, Smeland E B, Kvaloy S, Holte H, Delabie J, Connors J M, Lansdorp P M, Ouyang Q, Lister T A, Davies A J, Norton A J, Muller-Hermelink H K, Ott G, Campo E, Montserrat E, Wilson W H, Jaffe E S, Simon R, Yang L, Powell J, Zhao H, Goldschmidt N, Chiorazzi M, Staudt L M. Prediction of survival in follicular lymphoma based on molecular features of tumor-infiltrating immune cells. N Engl J Med. 2004 Nov. 18; 351(21):2159-69.

1-7. (canceled)

8: A method of treating non-Hodgkin's lymphoma in a subject, said method comprising the steps of:
a) assaying a sample which is tumor tissue or tumor cells of said subject by a stratification method wherein the expression levels of genes and gene signatures (patterns) are determined by Affymetrix array, RT-PCR, RNAseq, nanostrings, or RNAscope;
b) determining the presence of a stratification feature, which is an up-regulation of one or more genes which is (are) predictive of a response to copanlisib, or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18;
and wherein the degree of said up-regulation is statistically verified using the median weighted gene expression scores (WGS) for genes within the specific pathways and median gene expression signal level of Affymetrix array for single genes as cutoff values; and
c) administering a therapeutically effective amount of copanlisib, or a pharmacologically acceptable salt thereof, to the subject, wherein said subject has non-Hodgkin's lymphoma characterized by said stratification feature of up-regulation of one or more genes selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18.

9-11. (canceled)

12: A method for treatment or prophylaxis of non-Hodgkin's lymphoma in a subject, comprising administering a therapeutically effective amount of copanlisib, or a pharmacologically acceptable salt thereof, to the subject, wherein said subject has non-Hodgkin's lymphoma characterized by a stratification feature, wherein the stratification feature is up-regulation of one or more genes which is (are) predictive of a response to copanlisib, or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18.

13: The method according to claim 12, wherein said stratification feature which is an up-regulation of one or more genes which is (are) predictive of a response to copanlisib or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18;
is determined by a stratification method wherein the expression levels of said genes and gene signatures (patterns) are determined by Affymetrix array, RT-PCR, RNAseq, nanostrings, or RNAscope, and wherein the degree of said up-regulation is statistically verified using the median weighted gene expression scores (WGS) for genes within the specific pathways and median gene expression signal level of Affymetrix array for single genes as cutoff values.

14: The method according to claim 12, wherein said stratification feature which is an up-regulation of one or more genes which is (are) predictive of a response to copanlisib or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18;
has been determined positively in tumor tissue or tumor cells from said subject.

15. (canceled)

16: A method for treatment or prophylaxis of non-Hodgkin's lymphoma in a subject, comprising administering a therapeutically effective amount of a pharmaceutical combination comprising copanlisib, or a pharmacologically acceptable salt thereof, in combination with at least one or more further active substances, to the subject, wherein said subject has non-Hodgkin's lymphoma characterized by a stratification feature which is an up-regulation of one or more genes which is (are) predictive of a response to copanlisib, particularly copanlisib dihydrochloride, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18.

17: The method according to claim 16, wherein said stratification feature which is an up-regulation of one or more genes which is (are) predictive of a response to copanlisib or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18;
is determined by a stratification method wherein the expression levels of said genes and gene signatures (patterns) are determined by Affymetrix array, RT-PCR, RNAseq, nanostrings, RNAscope, and wherein the degree of said up-regulation is statistically verified using the median weighted gene expression scores (WGS) for genes within the specific pathways and median gene expression signal level of Affymetrix array for single genes as cutoff values.

18. (canceled)

19: The method according to claim 16, wherein said stratification feature which is an up-regulation of one or more genes which is (are) predictive of a response to copanlisib or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18;
has been determined positively in tumor tissue or tumor cells from said subject.

20-22. (canceled)

23: A method for treatment or prophylaxis of non-Hodgkin's lymphoma in a subject, comprising administering a therapeutically effective amount of a pharmaceutical formulation comprising copanlisib, or a pharmacologically acceptable salt thereof, in combination with an inert, nontoxic, and/or pharmaceutically suitable adjuvant to the subject, wherein said subject has non-Hodgkin's lymphoma characterized by a stratification feature which is an up-regulation of one or more genes which is (are) predictive of a response to copanlisib, or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18.

24: The method according to claim 23, wherein said stratification feature which is an up-regulation of one or more genes which is (are) predictive of a response to copanlisib, or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18;
is determined by a stratification method wherein the expression levels of said genes and gene signatures (patterns) are determined by Affymetrix array, RT-PCR, RNAseq, nanostrings, RNAscope, and wherein the degree of said up-regulation is statistically verified using the median weighted gene expression scores (WGS) for genes within the specific pathways and median gene expression signal level of Affymetrix array for single genes as cutoff values.

25: The method according to claim 23,
wherein said stratification feature which is an up-regulation of one or more genes which is (are) predictive of a response to copanlisib, or a pharmacologically acceptable salt thereof, and/or progression free survival, and which is (are) selected from the group consisting of:
BCR pathway genes: BLNK, BTK, CD19, CD22, CD40, CD69, CD72, CD79A, CD79B, LYN, MS4A1 (CD20), SYK, TNFRSF13B, TNFRSF17, FCGR2B;
PI3K pathway genes: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN; and
GPR18;
has been determined positively in tumor tissue or tumor cells from said subject.

26-29. (canceled)

30: A test for a gene expression signature measuring:
an up-regulation of one or more BCR pathway genes selected from: BLNK, BTK, CD19, CD22, CD40, CD69, CD72, CD79A, CD79B, LYN, MS4A1 (CD20), SYK, TNFRSF13B, TNFRSF17, FCGR2B,
to characterize BCR signaling in a subject to assess the suitability of said subject to a treatment with copanlisib, or a pharmacologically acceptable salt thereof,
said test being the measurement of said genes and gene signatures (patterns) using one of the following methods: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), it being possible for said cut off to be a median value generated from the tumors collected from the indication of the disease or established from a clinical trial evaluating the relationship between the expression level of a gene or a gene signature score and efficacy by the treatment of copanlisib, or a pharmacologically acceptable salt thereof.

31: A test for a gene expression signature measuring:
an up-regulation of one or more PI3K pathway genes selected from: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN,
to characterize PI3K signaling in a subject to assess the suitability of said subject to a treatment with copanlisib, or a pharmacologically acceptable salt thereof,
said test being the measurement of said genes and gene signatures (patterns) using one of the following methods: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), it being possible for said cut off to be a median value generated from the tumors collected from the indication of the disease or established from a clinical trial evaluating the relationship between the expression level of a gene or a gene signature score and efficacy by the treatment of copanlisib, or a pharmacologically acceptable salt thereof.

32: A test for a gene expression signature measuring:
an up-regulation of one or more BCR pathway genes selected from: BLNK, BTK, CD19, CD22, CD40, CD69, CD72, CD79A, CD79B, LYN, MS4A1 (CD20), SYK, TNFRSF13B, TNFRSF17, FCGR2B;
and
an up-regulation of one or more PI3K pathway genes selected from: AKT1, AKT3, GAB1, PIK3AP1, PIK3C3, PIK3CA, PIK3CB, PIK3CD, PIK3CG, PIK3IP1, PIK3R1, PIK3R2, PIK3R3, PIK3R4, PIK3R5, PTEN,
to characterize BCR and PI3K signaling in a subject to assess the suitability of said subject to a treatment with copanlisib, or a pharmacologically acceptable salt thereof,
said test being the measurement of said genes and gene signatures (patterns) using one of the following methods: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), it being possible for said cut off to be a median value generated from the tumors collected from the indication of the disease or established from a clinical trial evaluating the relationship between the expression level of a gene or a gene signature score and efficacy by the treatment of copanlisib, or a pharmacologically acceptable salt thereof.

33: A kit for the selection of a subject suffering from non-Hodgkin's lymphoma
characterized by and determining a stratification feature according to claim 30.

34: The kit according to claim 33, wherein said treatment is a monotherapy or combination therapy.

35: The kit according to claim 33, wherein said stratification feature is determined in a sample of tumor tissue or tumor cells from said subject.

36: A test for a gene expression signature measuring:
an up-regulation of one or more genes selected from: AKT1, AKT3, BLNK, BTK, CD19, CD69, CD72, CD79B, CD8B, FCGR2B, GAB1, LYN, MS4A1, PIK3AP1, PIK3C3, PIK3CA, PIK3CB, PIK3CD, PIK3CG, PIK3R1, PIK3R3, PIK3R4, PIK3R5, PTEN, TLR4, TNFRSF13B, VIM, GPR18,
in a subject to assess the suitability of said subject to a treatment with copanlisib, or a pharmacologically acceptable salt thereof,
said test being the measurement of said genes and gene signatures (patterns) using one of the following methods: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), it being possible for said cut off to be a median value generated from the tumors collected from the indication of the disease or established from a clinical trial evaluating the relationship between the expression level of a gene or a gene signature score and efficacy by the treatment of copanlisib, or a pharmacologically acceptable salt thereof.

37: A test for a gene expression signature measuring:
a down-regulation of one or more genes selected from: CD4, CD22, VCAN, TNFRSF17, NDE1, ICAM1, PIK3IP1, NFKBIA, MMP9, CD40, IL4I1, AKT2, CD79A, JAK3, MET, COL1A1, VWF, VEGFA, SPARC, FN1, SGK1, MT2A, IL1B, CD93, STAT5A, KDR, THBS1, LUM, S100A8, SERPINH1, BATF, PTGIR, CSTB, ITGB2, S100A9, SYK, CD14, CEBPB, SPHK1, NOP10, THBS2, COL5A2,
in a subject to assess the suitability of said subject to a treatment with copanlisib, or a pharmacologically acceptable salt thereof, said test being the measurement of said genes and gene signatures (patterns) using one of the following methods: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), it being possible for said cut off to be a median value generated from the tumors collected from the indication of the disease or established from a clinical trial evaluating the relationship between the expression level of a gene or a gene signature score and efficacy by the treatment of copanlisib, or a pharmacologically acceptable salt thereof.

38: A test for a gene expression signature measuring:
an up-regulation of one or more genes selected from: AKT1, AKT3, BLNK, BTK, CD19, CD69, CD72, CD79B, CD8B, FCGR2B, GAB1, LYN, MS4A1, PIK3AP1, PIK3C3, PIK3CA, PIK3CB, PIK3CD, PIK3CG, PIK3R1, PIK3R3, PIK3R4, PIK3R5, PTEN, TLR4, TNFRSF13B, VIM, GPR18,
and
a down-regulation of one or more genes selected from: CD4, CD22, VCAN, TNFRSF17, NDE1, ICAM1, PIK3IP1, NFKBIA, MMP9, CD40, IL4I1, AKT2, CD79A, JAK3, MET, COL1A1, VWF, VEGFA, SPARC, FN1, SGK1, MT2A, IL1B, CD93, STAT5A, KDR, THBS1, LUM, S100A8, SERPINH1, BATF, PTGIR, CSTB, ITGB2, S100A9, SYK, CD14, CEBPB, SPHK1, NOP10, THBS2, COL5A2,
in a subject to assess the suitability of said subject to a treatment with copanlisib, or a pharmacologically acceptable salt thereof,
said test being the measurement of said genes and gene signatures (patterns) using one of the following methods: Affymetrix array, RT-PCR, RNAseq, nanostrings, RNAscope, the degree of said up-regulation (or high expression) being determined by comparing the expression level of a gene from the measurement or a score (for example WGS) calculated/derived from the expression levels of all genes in the signature or pathway, with a threshold value (also known as a cutoff), it being possible for said cut off to be a median value generated from the tumors collected from the indication of the disease or established from a clinical trial evaluating the relationship between the expression level of a gene or a gene signature score and efficacy by the treatment of copanlisib, or a pharmacologically acceptable salt thereof.

39: A kit for the selection of a subject suffering from non-Hodgkin's lymphoma
characterized by and determining a stratification feature according to claim 36.

40: The kit according to claim 39, wherein said treatment is a monotherapy or combination therapy.

41: The kit according to claim 39, wherein said stratification feature is determined in a sample of tumor tissue or tumor cells from said subject.

42: The method according to claim 12, wherein copanlisib, or a pharmacologically acceptable salt thereof, is copanlisib dihydrochloride.

43: The method according to claim 12, wherein the non-Hodgkin's lymphoma is relapsed/refractory, indolent or aggressive non-Hodgkin's lymphoma.

44: The method according to claim 43, wherein the relapsed/refractory, indolent or aggressive non-Hodgkin's lymphoma is selected from the group consisting of follicular lymphoma (FL), chronic lymphocytic leukemia (CLL), marginal zone lymphoma (MZL), diffuse large B-cell lymphoma (DLBCL), mantle cell lymphoma (MCL), transformed lymphoma (TL), and or peripheral T-cell lymphoma (PTCL).
